(19) United States
(12) Patent Application Publication
(10) Pub. No.: US 2026/0036563 A1
(43) Pub. Date: Feb. 5, 2026

(54) Method For Rapid Detection Of Microorganisms In Beverages

(71) Applicant: Swire Coca-Cola (China) Co., Ltd, Shanghai (CN)

(72) Inventors: Weigang Xu, Shanghai (CN); Xuecai He, Shanghai (CN); Jiang Yao, Shanghai (CN); Yanju Zheng, Shanghai (CN)

(21) Appl. No.: 18/885,313

(22) Filed: Sep. 13, 2024

(30) Foreign Application Priority Data
Jul. 30, 2024 (CN) 2024110331790

(51) Int. Cl. G01N 33/14; G01N 21/25

(52) U.S. Cl. CPC G01N 33/14; G01N 21/251

(57) ABSTRACT

A method for rapid detection of microorganisms in beverages, includes placing the beverage sample under constant temperature conditions of 30-35° C. for 48 hours, and then adding it to a culture bottle with a sensing hydrogel and culturing it for 2 days for colorimetric detection; the sensing hydrogel is composed of Schiff base Composed of ZIF-90, neutral red, sodium alginate, and calcium chloride, it can absorb the carbon dioxide produced by microorganisms and respire, thereby causing a significant color change, from yellow to red. The advantage of the invention is that it significantly shortens the detection time, and achieves more economical and efficient detection by simplifying the operating process and reducing equipment requirements. It is especially suitable for microbial safety monitoring in the food and beverage industry to ensure product quality and consumer safety.

### 1. TECHNICAL FIELD

The invention relates to the technical field of microorganism detection, and in particular to a method for rapid detection of microorganisms in beverages.

### 2. BACKGROUND ART

In the process of beverage production and quality control, it is crucial to ensure the microbial safety of products. However, there are many problems with existing microbial detection technologies, which limit their efficiency and accuracy in practical applications. Although traditional plate count and membrane filtration methods can provide accurate results, however, the detection time is long, and it usually takes several days to get the results. In addition, these methods are complicated to operate and require strict aseptic operation and professional technicians to operate, which not only increases labor costs but also increases the risk of errors. In addition, traditional methods may not be sensitive enough when detecting low concentrations of microorganisms, and it is difficult to distinguish different types of microorganisms.

Although rapid detection technologies such as immunoassays and nucleic acid amplification techniques can provide faster results, however, they have high false positive and false negative rates in complex matrices (such as beverages), mainly due to matrix interference and insufficient specificity of the detection system. In addition, these highly sensitive rapid detection technologies usually require expensive instruments and reagents, which may impose a high economic burden on small and medium-sized enterprises. The complexity of sample processing is also a problem. Many rapid detection technologies require complex sample pretreatment steps to remove interfering substances and concentrate target microorganisms, which not only increases the number of operation steps, but may also introduce errors and variations. Therefore, there is an urgent need to further develop and optimize new detection methods to improve detection efficiency and accuracy, reduce costs, and simplify the operation process, so as to better meet the needs of the food and beverage industry for microbial testing and ensure product quality and safety.

### 3. SUMMARY OF THE INVENTION

In view of the above situation, in order to overcome the defects of the prior art, the invention provides a method for rapid detection of microorganisms in beverages.

In order to achieve the above purpose, the invention provides the following technical solutions: a method for rapid detection of microorganisms in beverages, including the following steps:
S1. place the beverage sample in a constant temperature incubator at 30-35° C. for 24 hours and set it as the sample to be tested;
S2. before starting the test, visually inspect the culture bottle and culture dish to prevent the use of culture bottle and culture dish that show signs of damage, leakage or deterioration; disinfect the outer surface of the culture bottle and culture dish with 75% alcohol and place in a sterile room under ultraviolet light for 30 minutes;
S3. put the sensing hydrogel into the bottom of the culture bottle sterilized in step S2, with a thickness of 1-2 cm, and clamp the culture dish 2-3 cm above the sensing hydrogel, leaving a gap next to it;
S4. before using the culture bottle, wipe the bottle mouth with a disposable sterile cotton swab, and then wipe off the residual moisture with a dry disposable cotton swab; use a disposable syringe to draw 5 mL of the sample to be tested prepared in step S1 and inject it into the culture dish, mark it as the test bottle, and set up a blank control bottle at the same time;
S5. place the test bottle prepared in step S4 and blank control bottle into an incubator, set the incubation temperature to 30° C., and control the ambient temperature to 25° C.; after 2 days of incubation, observe the color of the sensing hydrogel; if the sensing hydrogel changes from yellow to red, it means that microorganisms are present; if it remains yellow, it means that no microorganisms are present; the content of microorganisms is determined based on the degree of color change.

Preferably, the preparation method of the sensing hydrogel includes the following steps:
A1. dissolve imidazole-2-carboxaldehyde, zinc nitrate, and sodium formate in methanol by ultrasonic dispersion, transfer to a reactor and heat at 120° C. for 16-24 hours, cool to room temperature and centrifuge to obtain a precipitate; wash with ethanol and deionized water alternately three times and then vacuum dry to obtain ZIF-90;
A2. add the ZIF-90 obtained in step A1 into methanol, add anthranilic acid after uniform dispersion, heat for reflux at 60-80° C. for 12-24 hours, cool to room temperature, and centrifuge to obtain a precipitate; wash with ethanol and deionized water alternately three times and vacuum dry to obtain Schiff base ZIF-90;
A3. mix the Schiff base ZIF-90 obtained in step A2 with neutral red in distilled water, and after mixing evenly, adjust the pH to 10 and then add sodium alginate; after mixing evenly, add calcium chloride, and after cross-linking and curing, a sensing hydrogel is formed.

Preferably, in step A1, the usage ratio of imidazole-2-carboxaldehyde, zinc nitrate, sodium formate, and methanol is 2-4 mmol: 1 mmol: 1-2 mol: 40 mL.

Preferably, in step A2, the usage ratio of ZIF-90, methanol, and anthranilic acid is 50 mg: 6-10 mL: 1 mmol.

Preferably, in step A3, the usage ratio of Schiff base ZIF-90, neutral red, and distilled water is 50 mg: 10-30 mg: 50 mL, and the usage ratio of sodium alginate, calcium chloride, and distilled water is 1-1.5 g: 1-2 g: 50 mL.

The beneficial effects of the invention are as follows:

The invention significantly shortens the detection time, simplifies the operation process, and reduces the detection cost through an innovative method for rapid detection of microorganisms in beverages. Sensing hydrogel materials play a key role in this method. The sensing hydrogel can adsorb carbon dioxide and react with it to change color. This color-changing reaction can quickly indicate the presence of microorganisms. Microorganisms breathing in the petri dish produce carbon dioxide, and since carbon dioxide has a greater mass than air, it collects in the sensing hydrogel underneath the bottle. The introduction of Schiff base ZIF-90 further improves the adsorption capacity of the hydrogel for carbon dioxide, making the reaction between neutral red and carbon dioxide more obvious, making it easier to observe and judge the test results. Compared with the traditional plate counting method and membrane filtration method, this method shortens the detection time and greatly improves the detection efficiency. In addition, the hydrogel not only fixes the sensing solution, but also has a good carbon dioxide adsorption capacity. Based on the color change principle caused by the reaction of carbon dioxide and water to cause pH changes, the accuracy of the detection results is ensured. The material innovation of the sensing hydrogel enables it to perform well in capturing and adsorbing carbon dioxide. The efficient adsorption performance of the Schiff base ZIF-90 enhances the sensitivity and accuracy of the detection, and the obvious contrast between the neutral red and the carbon dioxide reaction color change more intuitively indicates the presence of microorganisms. This innovative material and method is not only economical and affordable, but also has high detection efficiency and accuracy, meeting the high requirements of the food and beverage industry for microbial detection and ensuring product quality and safety. In short, the invention has significant advantages in improving detection efficiency, simplifying operating procedures, reducing costs, improving sensitivity and accuracy, and can better meet the needs of the food and beverage industry for microbial detection.

### 4. BRIEF DESCRIPTION OF ACCOMPANY DRAWINGS

In order to explain the embodiments of the invention or the technical solutions in the prior art more clearly, the drawings that need to be used in the description of the embodiments or the prior art will be introduced hereinafter. Obviously, the drawings in the following description are only some embodiments of the invention. For those of ordinary skill in the art, other drawings may be obtained from these drawings without creative efforts.

FIG. 1 is a schematic diagram of the structure of the culture bottle, the culture dish, and the sensing hydrogel according to the invention;

FIG. 2 is a UV-visible absorption spectrum of the neutral red solution under alkaline conditions and after CO₂ is introduced;

FIG. 3 is a TEM image of the Schiff base ZIF-90 prepared in Embodiment 1 according to the invention;

FIG. 4 is an SEM image of the sensing hydrogel prepared in Embodiment 1 according to the invention;

FIG. 5 is a microbial concentration-grayscale fitting diagram obtained by the method described in in Embodiment 1 according to the invention;

FIG. 6 is a microbial concentration-grayscale fitting diagram obtained by the method described in in Comparative Embodiment 1 according to the invention;

FIG. 7 is a microbial concentration-grayscale fitting diagram obtained by the method described in in Comparative Embodiment 2 according to the invention;

In the figures: 1 refers to the culture bottle; 2 refers to the culture dish; 3 refers to the sensing hydrogel.

### 5. SPECIFIC EMBODIMENT OF THE INVENTION

The invention will be further described in detail hereinafter with reference to the embodiments and comparative embodiments, but it should not be understood as the scope of the invention being limited to the following embodiments, and all technologies implemented based on the above contents of the invention shall all fall within the protection scope of the invention.

The experimental methods in the following embodiments are conventional methods unless otherwise specified; the experimental materials used in the following embodiments and comparative embodiments are purchased from commercial channels unless otherwise specified.

Embodiment 1: the embodiment provides a method for rapid detection of microorganisms in beverages, including the following steps:
S1. place the beverage sample in a constant temperature incubator at 30° C. for 24 hours and set it as the sample to be tested;
S2. before starting the test, visually inspect the culture bottle 1 and culture dish 2 to prevent the use of culture bottle 1 and culture dish 2 that show signs of damage, leakage or deterioration; disinfect the outer surface of the culture bottle 1 and culture dish 2 with 75% alcohol and place in a sterile room under ultraviolet light for 30 minutes;
S3. put the sensing hydrogel 3 into the bottom of the culture bottle 1 sterilized in step S2, with a thickness of 2 cm, and clamp the culture dish 2 3 cm above the sensing hydrogel 3, leaving a gap next to it;
S4. before using the culture bottle 1, wipe the bottle mouth with a disposable sterile cotton swab, and then wipe off the residual moisture with a dry disposable cotton swab; use a disposable syringe to draw 5 mL of the sample to be tested prepared in step S1 and inject it into the culture dish 2, mark it as the test bottle, and set up a blank control bottle at the same time;
S5. place the test bottle prepared in step S4 and blank control bottle into an incubator, set the incubation temperature to 30° C., and control the ambient temperature to 25° C.; after 2 days of incubation, observe the color of the sensing hydrogel 3; if the sensing hydrogel 3 changes from yellow to red, it means that microorganisms are present; if it remains yellow, it means that no microorganisms are present; the content of microorganisms is determined based on the degree of color change.

The preparation method of the sensing hydrogel 3 includes the following steps:
A1. dissolve 4 mmol of imidazole-2-carboxaldehyde, 1 mmol of zinc nitrate, and 1 mmol of sodium formate in 40 mL of methanol by ultrasonic dispersion, transfer to a reactor and heat at 120° C. for 24 hours, cool to room temperature and centrifuge to obtain a precipitate; wash with ethanol and deionized water alternately three times and then vacuum dry to obtain ZIF-90;
A2. add 50 mg of the ZIF-90 obtained in step A1 into 10 mL of methanol, add 1 mmol of anthranilic acid after uniform dispersion, heat for reflux at 80° C. for 12 hours, cool to room temperature, and centrifuge to obtain a precipitate; wash with ethanol and deionized water alternately three times and vacuum dry to obtain Schiff base ZIF-90;
A3. mix 50 mg of the Schiff base ZIF-90 obtained in step A2 with 20 mg of neutral red in 50 mL of distilled water, and after mixing evenly, adjust the pH to 10 and then add 1 g of sodium alginate; after mixing evenly, add 1.2 g of calcium chloride, and after cross-linking and curing, a sensing hydrogel 3 is formed.

Embodiment 2: the embodiment provides a method for rapid detection of microorganisms in beverages, including the following steps:
S1. place the beverage sample in a constant temperature incubator at 35° C. for 24 hours and set it as the sample to be tested;
S2. before starting the test, visually inspect the culture bottle 1 and culture dish 2 to prevent the use of culture bottle 1 and culture dish 2 that show signs of damage, leakage or deterioration; disinfect the outer surface of the culture bottle 1 and culture dish 2 with 75% alcohol and place in a sterile room under ultraviolet light for 30 minutes;
S3. put the sensing hydrogel 3 into the bottom of the culture bottle 1 sterilized in step S2, with a thickness of 1 cm, and clamp the culture dish 2 3 cm above the sensing hydrogel 3, leaving a gap next to it;
S4. before using the culture bottle 1, wipe the bottle mouth with a disposable sterile cotton swab, and then wipe off the residual moisture with a dry disposable cotton swab; use a disposable syringe to draw 5 mL of the sample to be tested prepared in step S1 and inject it into the culture dish 2, mark it as the test bottle, and set up a blank control bottle at the same time;
S5. place the test bottle prepared in step S4 and blank control bottle into an incubator, set the incubation temperature to 30° C., and control the ambient temperature to 25° C.; after 2 days of incubation, observe the color of the sensing hydrogel 3; if the sensing hydrogel 3 changes from yellow to red, it means that microorganisms are present; if it remains yellow, it means that no microorganisms are present; the content of microorganisms is determined based on the degree of color change.

The preparation method of the sensing hydrogel 3 includes the following steps:
A1. dissolve 2 mmol of imidazole-2-carboxaldehyde, 1 mmol of zinc nitrate, and 1 mmol of sodium formate in 40 mL of methanol by ultrasonic dispersion, transfer to a reactor and heat at 120° C. for 16 hours, cool to room temperature and centrifuge to obtain a precipitate; wash with ethanol and deionized water alternately three times and then vacuum dry to obtain ZIF-90;
A2. add 50 mg of the ZIF-90 obtained in step A1 into 6 mL of methanol, add 1 mmol of anthranilic acid after uniform dispersion, heat for reflux at 60° C. for 24 hours, cool to room temperature, and centrifuge to obtain a precipitate; wash with ethanol and deionized water alternately three times and vacuum dry to obtain Schiff base ZIF-90;
A3. mix 50 mg of the Schiff base ZIF-90 obtained in step A2 with 10 mg of neutral red in 50 mL of distilled water, and after mixing evenly, adjust the pH to 10 and then add 1.5 g of sodium alginate; after mixing evenly, add 2 g of calcium chloride, and after cross-linking and curing, a sensing hydrogel 3 is formed.

Embodiment 3: the embodiment provides a method for rapid detection of microorganisms in beverages, including the following steps:
S1. place the beverage sample in a constant temperature incubator at 32° C. for 24 hours and set it as the sample to be tested;
S2. before starting the test, visually inspect the culture bottle 1 and culture dish 2 to prevent the use of culture bottle 1 and culture dish 2 that show signs of damage, leakage or deterioration; disinfect the outer surface of the culture bottle 1 and culture dish 2 with 75% alcohol and place in a sterile room under ultraviolet light for 30 minutes;
S3. put the sensing hydrogel 3 into the bottom of the culture bottle 1 sterilized in step S2, with a thickness of 2 cm, and clamp the culture dish 2 3 cm above the sensing hydrogel 3, leaving a gap next to it;
S4. before using the culture bottle 1, wipe the bottle mouth with a disposable sterile cotton swab, and then wipe off the residual moisture with a dry disposable cotton swab; use a disposable syringe to draw 5 mL of the sample to be tested prepared in step S1 and inject it into the culture dish 2, mark it as the test bottle, and set up a blank control bottle at the same time;
S5. place the test bottle prepared in step S4 and blank control bottle into an incubator, set the incubation temperature to 30° C., and control the ambient temperature to 25° C.; after 2 days of incubation, observe the color of the sensing hydrogel 3; if the sensing hydrogel 3 changes from yellow to red, it means that microorganisms are present; if it remains yellow, it means that no microorganisms are present; the content of microorganisms is determined based on the degree of color change.

The preparation method of the sensing hydrogel 3 includes the following steps:
A1. dissolve 3 mmol of imidazole-2-carboxaldehyde, 1 mmol of zinc nitrate, and 2 mmol of sodium formate in 40 mL of methanol by ultrasonic dispersion, transfer to a reactor and heat at 120° C. for 18 hours, cool to room temperature and centrifuge to obtain a precipitate; wash with ethanol and deionized water alternately three times and then vacuum dry to obtain ZIF-90;
A2. add 50 mg of the ZIF-90 obtained in step A1 into 8 mL of methanol, add 1 mmol of anthranilic acid after uniform dispersion, heat for reflux at 70° C. for 12 hours, cool to room temperature, and centrifuge to obtain a precipitate; wash with ethanol and deionized water alternately three times and vacuum dry to obtain Schiff base ZIF-90;
A3. mix 50 mg of the Schiff base ZIF-90 obtained in step A2 with 30 mg of neutral red in 50 mL of distilled water, and after mixing evenly, adjust the pH to 10 and then add 1.2 g of sodium alginate; after mixing evenly, add 1 g of calcium chloride, and after cross-linking and curing, a sensing hydrogel 3 is formed.

Comparative Embodiment 1: the comparative embodiment provides a method for rapid detection of microorganisms in beverages, which differs from Embodiment 1 only in that the Schiff base ZIF-90 is not added, and the remaining components, component contents, and experimental steps are the same as those in Embodiment 1.

Comparative Embodiment 2: the comparative embodiment provides a method for rapid detection of microorganisms in beverages, which differs from Embodiment 1 only in that sodium alginate and calcium chloride are not added, and the remaining components, component contents, and experimental steps are the same as those in Embodiment 1.

Experimental Embodiment 1: the UV-visible absorption spectra of neutral red solution and after CO₂ is introduced at pH 10 are measured using a UV-visible spectrophotometer.

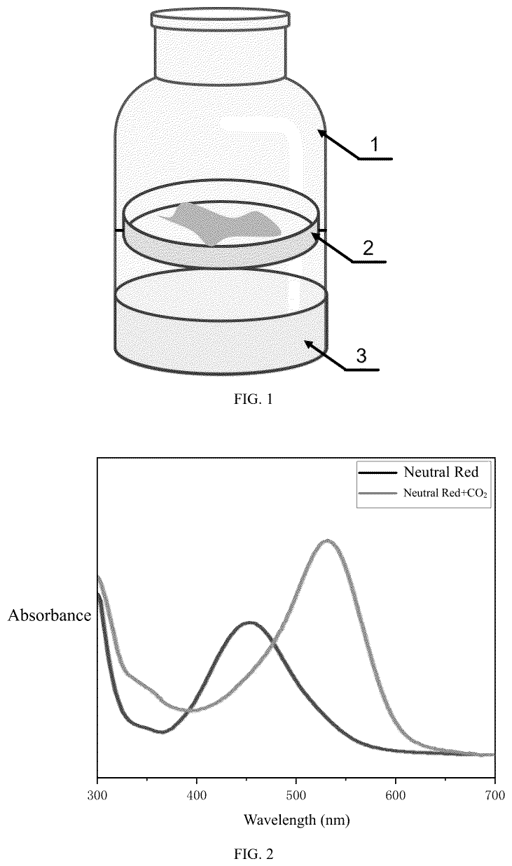

FIG. 2 is a UV-visible absorption spectrum of the neutral red solution under alkaline conditions and after CO₂ is introduced. As shown in the figure, a new absorption peak appeared at 533 nm after the introduction of CO₂ into the solution, and the solution changed from yellow to red, indicating that neutral red reacted with CO₂ in water.

Experimental Embodiment 2: observe the surface morphology of the Schiff base ZIF-90 prepared in Embodiment 1 using a projection electron microscope, and observe the microscopic morphology of the sensing hydrogel 3 prepared in Embodiment 1 using a scanning electron microscope.

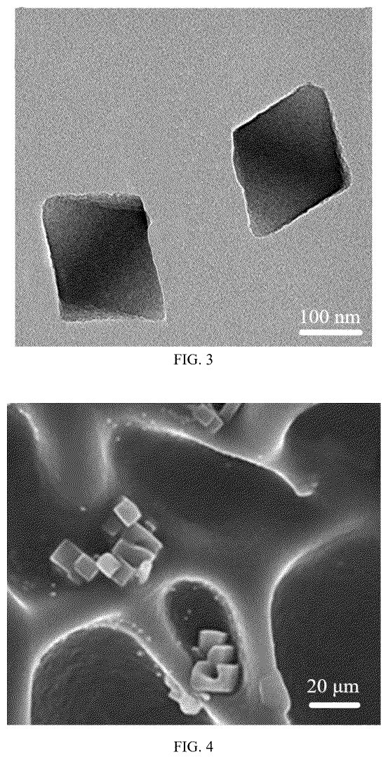

FIG. 3 is a TEM image of the Schiff base ZIF-90 prepared in Embodiment 1 according to the invention. As shown in the figure, the Schiff base ZIF-90 still maintains a cubic morphology and the structure is not destroyed. FIG. 4 is a SEM image of the sensing hydrogel prepared in Embodiment 1 according to the invention. As shown in the figure, Schiff base ZIF-90 is embedded in the three-dimensional cross-linked structure of the hydrogel, indicating that the sensing hydrogel 3 of the invention is successfully prepared.

Experimental Embodiment 3: mobile phone-assisted standard colorimetric quantitative detection.

Prepare Escherichia coli samples of different concentrations and culture according to the methods described in Embodiment 1, Comparative Embodiment 1, and Comparative Embodiment 2, respectively. After the culture were completed, observe the color change of the sensing hydrogel 3, and take photos under a fixed light source using a mobile phone. Perform image processing using ImageJ software, and convert the obtained RGB values into grayscale values, which are fitted and analyzed with the microbial concentration.

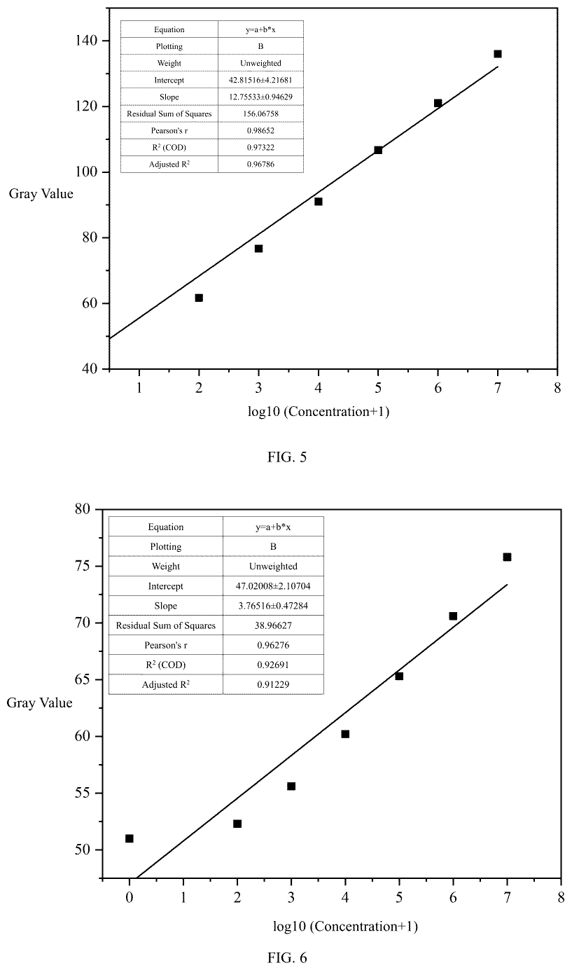

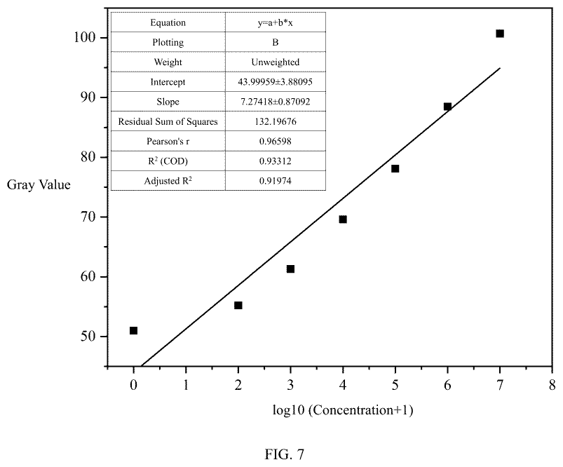

FIG. 5 is a microbial concentration-grayscale fitting diagram obtained by the method described in in Embodiment 1 according to the invention. As shown in the figure, when the microbial concentration is in the range of 10²-10⁷, the gray value=42.8+12.8 ln (concentration+1), R²=0.97, which has good sensitivity and good fit. FIG. 6 is a microbial concentration-grayscale fitting diagram obtained by the method described in in Comparative Embodiment 1 according to the invention. As shown in the figure, when the microbial concentration is in the range of 10²-10⁷, the gray value=47.0+3.76 ln (concentration+1), R²=0.91, and both the sensitivity and fit are poor. FIG. 7 is a microbial concentration-grayscale fitting diagram obtained by the method described in in Comparative Embodiment 2 according to the invention. As shown in the figure, when the microbial concentration is in the range of 10²-10⁷, the gray value=44.0+7.26 ln (concentration+1), R²=0.92, and the sensitivity and fit are between Embodiment 1 and the Comparative Embodiment 1. The above analysis shows that the introduction of Schiff base ZIF-90 and hydrogel can improve the sensitivity of neutral red to carbon dioxide, as well as the stability of detection and improve accuracy.

The invention and the embodiments thereof are described hereinabove, and this description is not restrictive. What is shown in the drawings is only one of the embodiments of the invention, and the actual structure is not limited thereto. All in all, structural methods and embodiments similar to the technical solution without deviating from the purpose of the invention made by those of ordinary skill in the art without creative design shall all fall within the protection scope of the invention.

1. A method for rapid detection of microorganisms in beverages, including following steps:
S1. place the beverage sample in a constant temperature incubator at 30-35° C. for 24 hours and set it as the sample to be tested;
S2. before starting the test, visually inspect the culture bottle and culture dish to prevent the use of culture bottle and culture dish that show signs of damage, leakage or deterioration; disinfect the outer surface of the culture bottle and culture dish with 75% alcohol and place in a sterile room under ultraviolet light for 30 minutes;
S3. put the sensing hydrogel into the bottom of the culture bottle sterilized in step S2, with a thickness of 1-2 cm, and clamp the culture dish 2-3 cm above the sensing hydrogel, leaving a gap next to it;
S4. before using the culture bottle, wipe the bottle mouth with a disposable sterile cotton swab, and then wipe off the residual moisture with a dry disposable cotton swab; use a disposable syringe to draw 5 mL of the sample to be tested prepared in step S1 and inject it into the culture dish, mark it as the test bottle, and set up a blank control bottle at the same time;
S5. place the test bottle prepared in step S4 and blank control bottle into an incubator, set the incubation temperature to 30° C., and control the ambient temperature to 25° C.; after 2 days of incubation, observe the color of the sensing hydrogel; if the sensing hydrogel changes from yellow to red, it means that microorganisms are present; if it remains yellow, it means that no microorganisms are present; the content of microorganisms is determined based on the degree of color change.

2. The method for rapid detection of microorganisms in beverages of claim 1, wherein the preparation method of the sensing hydrogel includes the following steps:
A1. dissolve imidazole-2-carboxaldehyde, zinc nitrate, and sodium formate in methanol by ultrasonic dispersion, transfer to a reactor and heat at 120° C. for 16-24 hours, cool to room temperature and centrifuge to obtain a precipitate; wash with ethanol and deionized water alternately three times and then vacuum dry to obtain ZIF-90;
A2. add the ZIF-90 obtained in step A1 into methanol, add anthranilic acid after uniform dispersion, heat for reflux at 60-80° C. for 12-24 hours, cool to room temperature, and centrifuge to obtain a precipitate; wash with ethanol and deionized water alternately three times and vacuum dry to obtain Schiff base ZIF-90;
A3. mix the Schiff base ZIF-90 obtained in step A2 with neutral red in distilled water, and after mixing evenly, adjust the pH to 10 and then add sodium alginate; after mixing evenly, add calcium chloride, and after cross-linking and curing, a sensing hydrogel is formed.

3. The method for rapid detection of microorganisms in beverages of claim 2, wherein in step A1, the usage ratio of imidazole-2-carboxaldehyde, zinc nitrate, sodium formate, and methanol is 2-4 mmol: 1 mmol: 1-2 mol: 40 mL.

4. The method for rapid detection of microorganisms in beverages of claim 3, wherein in step A2, the usage ratio of ZIF-90, methanol, and anthranilic acid is 50 mg: 6-10 mL: 1 mmol.

5. The method for rapid detection of microorganisms in beverages of claim 4, wherein in step A3, the usage ratio of Schiff base ZIF-90, neutral red, and distilled water is 50 mg: 10-30 mg: 50 mL, and the usage ratio of sodium alginate, calcium chloride, and distilled water is 1-1.5 g: 1-2 g: 50 mL.
